(19) United States
(12) Patent Application Publication
(10) Pub. No.: US 2023/0398000 A1
(43) Pub. Date: Dec. 14, 2023

(54) TOOL FOR INTER OPERATIVE USE DURING DIRECT ANTERIOR APPROACH TOTAL HIP ARTHROPLASTY AND RELATED METHODS

(71) Applicant: MATHYS AG BETTLACH, Bettlach (CH)

(72) Inventor: Patrick McDougall FELL, Auckland (NZ)

(21) Appl. No.: 18/249,213

(22) PCT Filed: Oct. 15, 2021
(86) PCT No.: PCT/IB2021/059486

§ 371 Date: Apr. 14, 2023

(30) Foreign Application Priority Data
Oct. 16, 2020 (NZ) 769068

(51) Int. Cl. A61F 2/46; A61B 17/17

(52) U.S. Cl. CPC A61F 2/4657; A61B 17/1742; A61F 2002/4687; A61F 2002/4658; A61F 2002/4668

(57) ABSTRACT

A surgical guide tool to visually aid in setting of the desired leg length and femoral offset of a patient inter-operatively during direct anterior approach arthroplasty. The tool comprises a femur contact member able to make at least two point contact with the femur and a pelvis contact member able to make at least one point contact with the pelvis. The femur contact member and the pelvis contact member are coupled together. They can move relative to each other to allow them to contact the femur and pelvis respectively. They can also be made to lock relative to each other to be used as a guide for setting the leg length and femoral offset provided for by the hip prosthetic.

### FIELD OF INVENTION

The present invention relates to a tool for inter operative use during direct anterior approach total hip arthroplasty and method therefor. More particularly but not exclusively it relates to a non-invasive tool for achieving a repeat, post femoral head removal, of the leg length and femoral offset in direct anterior approach total hip arthroplasty.

### BACKGROUND

There are 4 common and different approaches through the soft tissues that surgeons may elect to use to access the hip joint for total hip arthroplasty. These are commonly known as the:
Posterior approach
Lateral approach,
Anterolateral approach, and
Anterior (known as Direct Anterior Approach)

Each approach offers different advantages and disadvantages and each presents the surgeon with a certain perspective of the hip joint.

Most surgeons adhere to just one approach in their daily practice, usually the one they have been most trained in or experienced in.

In many countries the posterior approach has been by far the most popular. Many surgeons are hence very familiar with the procedure, the visual landmarks, tools used and variables that can be encountered during a posterior approach procedure.

However, the direct anterior approach is gaining in popularity as patients experience less post-operative pain, earlier mobilisation and reduced hospital stays. The direct anterior approach is made toward the front of the hip, and so the view of the hip for the surgeon is quite different to the posterior approach. The same bony landmarks as are on view during the posterior approach are not on view during the direct anterior approach. Surgeons experienced with the posterior approach may hence not be that comfortable with the direct anterior approach and may require more guidance and validation from tools that help them achieve a successful outcome.

Once the femoral head has been removed, the desired LL, FO cannot be accurately restored without the assistance of some means of measuring the dimensional position of the trial and definitive femoral and acetabular components prosthetics with respect to each other.

When the femoral head is removed and prostheses introduced, it is important for the surgeon to position the prostheses such that the spatial arrangement of the femur and pelvis bones are in the same or desired place with respect to each other as they were pre-op. Hence during arthroplasty it is important that the prostheses are configured to ensure that the leg length (LL), femoral offset (FO) and anterior offset (AO) of the femur relative to the pelvis bone is set the same as before the surgery, or able to be corrected, for instance if arthritis in the joint has caused shortening of the overall leg length. The LL and FO dimensions and often also the AO dimension decouple inter operatively and a surgeon needs to be guided to ensure that the femur and pelvis are returned to position relative each other so that these dimensions are appropriately reset post op. This requires a correctly sized and shaped prosthetic to be correctly configured to the femur and pelvis. If they are not so returned problems can arise by virtue of the leg being longer or the lateral width of the new hip joint not matching that of the hip on the other side. Inter operatively detecting any change in LL, FO and preferably also AO, during final prosthetic configuration, will help guide a surgeon to make sure that any undesirable changes are reduced sufficiently or eliminated.

Various simple and effective methods exist inter operatively to detect change in LL, FO in the Posterior Approach, but there are no simple methods for detecting change in LL, FO and AO for the Direct Anterior Approach.

Preoperatively, for both posterior and anterior approaches, a common method is to make a measurement off an X-ray radiograph and then attempt to reproduce this measurement with a sterile ruler intraoperatively. However, this method can suffer inaccuracies because whilst distance can be correct, the angle may be wrong. There are also currently a number of computer-navigated practices, intraoperative imaging methods and also some devices. Some are more accurate or complicated than others. Instrumented methods are invasive. They require drilling into bone. They may also only work for hip replacements performed through the posterior approach so may not be successfully used in the direct anterior approach. Methods and technology available for ensuring the post-operative LL and FO are set correctly during the direct anterior approach can be time consuming, complicated, are costly and/or may involve intraoperative X-ray screening.

The AO cannot be calculated from an Anterior-Posterior directed radiograph. AO has traditionally not been measured, possibly because surgeons are comfortable in achieving it through correct femoral anteversion (the angle between the femoral neck and the femoral shaft in the transverse plane ie how much forward of the shaft the femoral head is, toward the front of the body). It could also be possible that it has not been measured because there has not been easy or reliable methods to do so.

It may therefore be an object of the present invention to provide a non-invasive surgical guide tool to visually show change in LL, FO and preferably also AO for use during direct anterior approach total hip arthroplasty that addresses the above mentioned problems and/or to provide the public with a useful choice.

It may also be an object of the present invention to provide a surgical guide tool for inter operative use during direct anterior approach total hip arthroplasty and method therefor that addresses the above mentioned problems and/or to provide the public with a useful choice.

It may also be an object of the present invention to provide a surgical guide tool for achieving, post surgery, a repeat of the pre surgical leg length, femoral offset and preferably also anterior offset in direct anterior approach hip replacement surgery that addresses the above mentioned problems and/or to provide the public with a useful choice.

It may also be an object of the present invention to provide a surgical guide tool to aid in hip and femur alignment and/or method therefore for direct anterior approach total hip arthroplasty that addresses the above mentioned problems and/or to provide the public with a useful choice.

It may also be an object of the present invention to provide a surgical guide tool for use inter operatively during direct anterior approach total hip arthroplasty for detecting change in LL, FO and preferably also AO, during final prosthetic configuration, to aid a surgeon in reducing or eliminating any undesirable changes to the pre-op LL, FO and AO and/or to provide the public with a useful choice.

It may also be an object of the present invention to provide a surgical guide tool for use inter operatively during direct anterior approach total hip arthroplasty for detecting change in LL, FO and preferably also AO, to aid in configuring the hip prosthetic to reduce or eliminate any undesirable changes to the pre-op LL, FO and AO and/or to provide the public with a useful choice.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

For the purpose of this specification, where method steps are described in sequence, the sequence does not necessarily mean that the steps are to be chronologically ordered in that sequence, unless there is no other logical manner of interpreting the sequence.

### BRIEF DESCRIPTION OF THE INVENTION

In a first aspect the present invention may be said to broadly consist a surgical guide tool to visually aid in setting of the desired leg length and femoral offset of a patient inter-operatively during direct anterior approach arthroplasty the tool comprising:
a. a femur contact member able to make at least two point contact with the femur at and/or inferior the femoral osteotomy cut, to set a position and at least one notional angle of the femur contact member on and relative the femur, and
b. a pelvis contact member able to make at least one point contact with the pelvis,
   where the femur contact member and the pelvis contact member are coupled together;
      (a) in manner able to move relative to each other to allow (i) said at least two point contact with the femur and (ii) said at least one point contact with the pelvis to be made, and
      (b) in a manner to lock relative movement of the pelvis contact member and femur contact member.

Preferably the at least one notional angle is an angle about an axis that extends normal to the coronal plane of the patient.

Preferably the at least one notional angle is an angle about an axis that extends normal to the transverse plane of the patient.

Preferably the at least one notional angle is an angle about an axis that extends normal to the sagittal plane of the patient.

Preferably the at least one notional angle is at least two angles being an angle about an axis that extends normal to the coronal plane of the patient an angle about an axis that extends normal to the transverse plane of the patient.

Preferably the at least one notional angle is at least two angles being an angle about an axis that extends normal to the coronal plane of the patient and an angle about an axis that extends normal to the sagittal plane of the patient.

Preferably the at least one notional angle is at least two angles being an angle about an axis that extends normal to the sagittal plane of the patient and an angle about an axis that extends normal to the transverse plane of the patient.

Preferably the at least one notional angle is three angles being an angle about an axis that extends normal to the coronal plane of the patient and an angle about an axis that extends normal to the transverse plane of the patient and an angle about an axis that extends normal to the sagittal plane of the patient.

Preferably said at least two point contact of the femur by the femur contact member is at at least two spaced apart references respectively, on or of the femur.

Preferably the references on or of the femur are selected from any two of:
a. a line marked on the femur at or inferior of the femoral osteotomy cut location,
b. at least one mark marked on the femur inferior of the femoral osteotomy cut location,
c. at least one other mark marked on the femur inferior of the femoral osteotomy cut location,
d. the face of the femur defined by the femoral osteotomy cut or its anterior cortex precursor, and
e. a natural landmark of the surface of the femur inferior of the femoral osteotomy cut location.

Preferably the references on or of the femur are (a) at least one mark marked on the femur inferior of the femoral osteotomy cut location, and (b) the face of the femur defined by the femoral osteotomy cut or its anterior cortex precursor.

Preferably the at least one mark marked on the femur is selected from a line, dot, cross or v shaped marking.

Preferably said at least one point contact of the pelvis contact member is at at least one reference on or of the pelvis.

Preferably the at least one reference on or of the pelvis is selected from one of:
a. a line marked on the pelvis,
b. a point marked on the pelvis, and
c. a natural landmark of the pelvis.

Preferably the at least two point contact for the femur contact member is provided by two spaced apart contact members of the femur contact member.

Preferably the femoral contact member comprises of a leg (herein after “femur leg”) having a first distal end at where the at least two point contact for the femur contact member directly or indirectly occurs.

Preferably the two spaced apart contact members are presented at the end of the femur leg.

Preferably a first of said two spaced apart contacts members is a pin.

Preferably the pin is defined by and at the end of the femur leg.

Preferably a second of said two spaced apart contact members is a plate having a surface or surfaces able to be held against the cut surface of the femur or the line mark of the femur to align therewith.

Preferably the plate is attached to the femur leg.

Preferably the plate is attached to the femur leg in a fixed manner.

Preferably the plate, when located against the cut surface or the line marking on the femur, sets a said notional angle of the femur contact member relative the femur.

Preferably the pin when set on a said mark of the femur sets a position of the femur contact member relative the femur.

Preferably the plate, when located against the cut surface or the line marking on the femur, and the pin, when set on a said mark of the femur, set a said notional angle and a position of the femur contact member relative the femur.

Preferably a second of said two contact members of the femur contact member is a pin to locate as a said mark on the femur.

Preferably the femur leg of the femur contact member comprises a second distal end opposite the first distal end.

Preferably the pelvis contact member is coupled directly or indirectly to the femur leg.

Preferably the pelvis contact member is coupled directly or indirectly to the femur leg in a manner able to move along yet be fastened to the femur leg.

Preferably the pelvis contact member is coupled directly or indirectly to the femur leg in a manner able to rotate about yet be fastened to the femur leg.

Preferably the pelvis contact member is coupled directly or indirectly to the femur leg in a manner able to move along yet be fastened to the femur leg and in a manner able to rotate about yet be fastened to the femur leg to prevent its rotations.

Preferably the pelvis contact member is coupled directly or indirectly to the femur leg intermediate of the second distal end and the first distal end of the femur leg.

Preferably the pelvis contact member is coupled directly to the femur leg in a manner be able to slide relative the femur leg between the first and second distal ends and be selectively fastened to the femur leg to prevent sliding.

Preferably the pelvis contact member comprises a leg (herein after “pelvis leg”) with a first distal end at where the at least one point contact of the pelvis contact member can be made with the pelvis.

Preferably the pelvis leg has a second distal end opposite the first distal end at where the pelvis leg is coupled to the femur leg.

Preferably the angle of the pelvis leg relative the femur leg can be changed and set.

Preferably the femur leg and the pelvis leg are coupled together (a) in manner able to move relative to each other to allow (i) said at least two point contact with the femur and (ii) said at least one point contact with the pelvis to be made and (b) in a manner to lock relative movement of the pelvis leg and femur leg.

Preferably the at least one point contact of the pelvis leg is able to be made by a pin of the pelvis leg.

Preferably the pin of the pelvis leg is at the first distal end of the pelvis leg.

Preferably the pin of the pelvis leg is able to move along a notional arc to rotate about the femur leg about a first axis of rotation.

Preferably the radius of the notional arc is able to be varied by rotating the pelvis leg about an axis transverse to the first axis of rotation.

Preferably the pelvis contact member and the femur contact member are coupled together (a) in manner able to move relative to each other to allow (i) said at least two point contact with the femur and (ii) said at least one point contact with the pelvis to be made and (b) in a manner to lock relative movement of the pelvis contact member and femur contact member.

Preferably the at least one point contact with the pelvis for the pelvis contact member is at the surface anterior inferior iliac spine.

Preferably the at least one point contact with the pelvis for the pelvis contact member is only at the surface anterior inferior iliac spine.

Preferably at least one of the at least two point contact of the femoral member is inferior of the femoral osteotomy cut location.

Preferably the tool is a non-invasive tool.

Preferably the tool is a non-invasive tool requiring no invasive anchoring of the femur contact member nor the pelvis contact member.

Preferably the tool is pre-operatively made and inter-operatively adjustable to change the distance between contact point of the pelvis contact member and the two contact points of the femur contact member.

Preferably the femur contact member is able to set a vector to a visual reference on the surface of the pelvis pre full femoral osteotomy from at least two spaced apart references on the surface of the femur.

In a further aspect the present invention may be said to be a surgical guide tool for interoperative use during direct anterior hip replacement surgery to aid in detecting a change in LL and FO at the surgical site during final prosthetic configuration to the patient, the tool comprising:
a. a femur contact member able to positionally and rotationally register on and with the femur by provision of at least two femur contact points of the femur contact member,
b. a pelvis contact member able to positionally register on and with the pelvis,
wherein the pelvis contact member and the femur contact member are (a) coupled together by a coupling in (a) a movable manner relative each other so that the pelvis contact member can positionally register with a reference on the pelvis when the femur contact member is positionally and rotationally registered with respective references of the femur prior to total femoral head removal and (b) a manner able to prevent movement between the pelvis contact member and the femur contact member so that after total femoral head removal the femur contact member can be returned to the surgical site and register at the references of the femur to visually detect change in LL and FO.

In a further aspect the present invention may be said to be a surgical guide tool for interoperative use during direct anterior hip replacement surgery at the surgical site to aid in setting LL and FO during final prosthetic configuration to the patient, the tool comprising:
a. a femur contact member able to positionally and rotationally register on and with the femur by provision of at least two femur contact points of the femur contact member,
b. a pelvis contact member able to positionally register on and with the pelvis,
wherein the pelvis contact member and the femur contact member are (a) coupled together by a coupling in (a) a movable manner relative each other so that the pelvis contact member can positionally register with a reference on the pelvis when the femur contact member is positionally and rotationally registered with respective references of the femur prior to total femoral head removal and (b) a manner able to prevent movement between the pelvis contact member and the femur contact member so that after total femoral head removal the femur contact member can be returned to the surgical site and register at the references of the femur to visually detect change in LL and FO.

The present invention may also be broadly be said to be an adjustable surgical guide tool for interoperative use during direct anterior hip replacement surgery to aid in setting post op LL and FO during final prosthetic configuration to the patient the tool able to set a 3 dimensional vector to a visual reference on the surface of the pelvis pre full femoral osteotomy from at least two spaced apart references on the surface of the femur the tool comprising
a. a femur contact member able to make at least one point contact with the femur at and/or inferior the femoral osteotomy cut, and
b. a pelvis contact member able to make at least one point contact with the pelvis,
   i. wherein between the femur contact member and pelvis contact member a total of at least 3 of said at least one points of contact are made, and
   ii. wherein the femur contact member and the pelvis contact member are coupled together;
   (a) in manner able to move relative to each other to allow (i) said at least one point contact with the femur and (ii) said at least one point contact with the pelvis to be made, and
   (b) in a manner to lock relative movement of the pelvis contact member and femur contact member to set a position and a notional angle of the femur contact member on and relative the femur and set a position and a notional angle of the pelvis contact member with the pelvis.

In yet a further aspect the present invention may be said to be an adjustable surgical guide tool for interoperative use during direct anterior hip replacement surgery to aid in setting the post op LL and FO during final prosthetic configuration to the patient, the tool able to set a 3 dimensional vector to a visual reference on the surface of the pelvis pre full femoral osteotomy from at least two spaced apart references on the surface of the femur the tool comprising;
a. a femur contact member having a pin to contact one of the two spaced apart references of the surface of the femur and one of a plate and second pin to contact the other of the two spaced apart references of the femur,
b. a pelvis contact member having a pin to contact the visual reference on the surface of the pelvis.

Preferably the tool is to visually aid in re-setting of the pre-operative LL and FO of a patient inter-operatively during direct anterior approach arthroplasty.

Preferably the desired leg length and femoral offset is the same as the pre-operative leg length and femoral offset.

Preferably the tool also aids in setting the AO.

The present invention may also broadly be said to be a method of intraoperatively detecting a change in LL and FO at the surgical site during direct anterior hip replacement surgery to aid in during final prosthetic configuration to the patient, the method comprising:
a. positionally and rotationally registering a femur contact member on and with the femur at least two spaced apart references of or on the femur,
b. positionally registering at pelvis contact member on and with the pelvis on and at a pelvis reference,
c. locking the pelvis contact member and femur contact member with each other (directly or indirectly) to prevent their relative movement,
d. removing the femur contact member and pelvis contact member in their locked condition,
e. removing the femoral head, and
f. prior to setting the definitive prosthetic to the patient, re-introducing the femur contact member and pelvis contact member by registering the femur contact member to the at least two spaced apart references of or on the femur and observing the location of the pelvis contact member relative the pelvis.

The present invention may also be said to be a method of intraoperatively aiding the setting of the desired LL and FO during direct anterior hip replacement surgery, the method comprising:
a. positionally and rotationally registering a femur contact member on and with the femur at least two spaced apart references of or on the femur,
b. positionally registering at pelvis contact member on and with the pelvis on and at a pelvis reference,
c. locking the pelvis contact member and femur contact member with each other (directly or indirectly) to prevent their relative movement,
d. removing the femur contact member and pelvis contact member in their locked condition,
e. removing the femoral head,
f. prior to setting the definitive prosthetic to the patient, re-introducing the femur contact member and pelvis contact member by registering the femur contact member to the at least two spaced apart references of or on the femur and observing the location of the pelvis contact member relative the pelvis, and
g. setting the definitive prosthetic to the patient.

Preferably the present invention may also be said to reside in the use of a tool as herein described during direct anterior approach total hip arthroplasty.

In yet a further aspect the present invention may be said to be a surgical guide tool to visually aid in setting of the leg length and femoral offset of a patient inter-operatively during direct anterior approach arthroplasty the tool comprising:
a. a femur contact member able to make at least one point contact with the femur at and/or inferior the femoral osteotomy cut, and
b. a pelvis contact member able to make at least one point contact with the pelvis,
   i. wherein between the femur contact member and pelvis contact member a total of at least 3 of said at least one points of contact are made, and
   ii. wherein the femur contact member and the pelvis contact member are coupled together;
   (a) in manner able to move relative to each other to allow (i) said at least one point contact with the femur and (ii) said at least one point contact with the pelvis to be made, and
   (b) in a manner to lock relative movement of the pelvis contact member and femur contact member to set a position and a notional angle of the femur contact member on and relative the femur and set a position and a notional angle of the pelvis contact member with the pelvis.

In yet a further aspect the present invention may be said to be a surgical guide tool to visually aid in setting of the leg length and femoral offset of a patient inter-operatively during direct anterior approach arthroplasty the tool comprising:
a. a femur contact member able to make at least one point contact with the femur at and/or inferior the femoral osteotomy cut, and
b. a pelvis contact member able to make at least one point contact with the pelvis,
   i. wherein the femur contact member and pelvis contact member provide for at least 3 of said at least one points of contact to be made with the femur and pelvis
   ii. wherein the femur contact member and the pelvis contact member are coupled together;
      (a) in manner able to move relative to each other to allow (i) said at least one point contact with the femur and (ii) said at least one point contact with the pelvis to be made, and
      (b) in a manner to lock relative movement of the pelvis contact member and femur contact member, to set a position and a notional angle of the femur contact member on and relative the femur and set a position and a notional angle of the pelvis contact member with the pelvis.

In yet a further aspect the present invention may be said to be a surgical guide tool to visually aid in setting of the leg length and femoral offset of a patient inter-operatively during direct anterior approach arthroplasty the tool comprising:
a. a femur contact member able to make contact with the femur at and/or inferior the femoral osteotomy cut, and
b. a pelvis contact member able to make contact with the pelvis,
wherein the femur contact member and the pelvis contact member are coupled together in manner able to:
   (a) move relative to each other to allow both said femur contact member to make contact with the femur and said pelvis contact member to make contact with said pelvis in a tri-pod manner,
   (b) lock relative to each other to set a position and a notional angle of the femur contact member on and relative the femur and set a position and a notional angle of the pelvis contact member with the pelvis.

Other aspects of the invention may become apparent from the following description which is given by way of example only and with reference to the accompanying drawings.

As used herein the term “and/or” means “and” or “or”, or both.

As used herein “(s)” following a noun means the plural and/or singular forms of the noun.

The term “comprising” as used in this specification [and claims] means “consisting at least in part of”. When interpreting statements in this specification [and claims] which include that term, the features, prefaced by that term in each statement, all need to be present but other features can also be present. Related terms such as “comprise” and “comprised” are to be interpreted in the same manner.

The entire disclosures of all applications, patents and publications, cited above and below, if any, are hereby incorporated by reference.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.)

### BRIEF DESCRIPTION OF THE DRAWINGS

The invention will now be described by way of examples only and with reference to the drawings in which:

FIG. 1 is an anterior view of a human skeleton,

FIG. 2 is a view of a human showing the transverse plane k, the coronal plane m, and the sagittal plane e and corresponding axes,

FIG. 3 is an x-ray of a hip region of a person seen from an anterior direction,

FIG. 4 is a view of part of the pelvis and femur at a hip region of a person,

FIG. 5 shows a post-op view of the region of FIG. 4 with a hip prosthetic in place,

FIG. 6 is an x-ray image of a hip region of a person with a hip prosthetic shown in overlay,

FIG. 7 is a side view of a preferred form of the surgical guide tool of the present invention,

FIG. 8 is a front view of the tool of FIG. 7,

FIG. 8A shows the tool in schematic and with reference a 3D reference frame with orthogonal axes to illustrate the tool's reconfigurability,

FIG. 9 is an exploded view of the tool of FIG. 7,

FIG. 10 shows a view of the hip region with a femoral osteotomy cut having been partially made into the anterior cortex of the femur,

FIG. 11 shows the tool registered with the femur and pelvis bone and pre femoral head removal, showing the tool making contact with the references of the femur and the pelvis bone for vector setting of the tool,

FIG. 12 is a close-up view of a region of FIG. 11,

FIG. 13 shows the view of FIG. 11 in an alternative direction,

FIG. 14 shows the tool being reused, after initial vector setting of the tool prior to final hip prosthetic configuration, to detect any undesirable change in the spatial relationship between the references of the femur and the pelvis,

FIG. 15 is a view of part of the tool and the face of the cut made to remove the femoral head,

FIG. 16 shows the hip region with the prosthetic in situ and the reference markings,

FIG. 17 shows the hip region as per FIG. 16 and including a gauge to visually show any change between the tool set vector and the reference markings,

FIG. 18 shows a perspective view of part of the femur and a tool according to the present invention having an alternative configuration,

FIG. 19 is a front view of part of the tool of FIG. 18

FIG. 20 is a perspective view of part of the tool of FIG. 18,

FIG. 21 is an end view of part of the tool of FIG. 18,

FIG. 22 shows yet an alternative variation of tool of the present invention,

FIG. 23 shows part of the tool in position relative to the femur,

FIG. 24 is a table illustrating examples of variation of the tool in relation to the femur (F) and pelvis (P),

FIG. 24A shows one example from the table of FIG. 24 in more detail,

FIG. 25 shows a variation to the plate of the femur contact member of the tool, and

FIG. 26 shows a further variation of a tool of the present invention.

### DETAILED DESCRIPTION

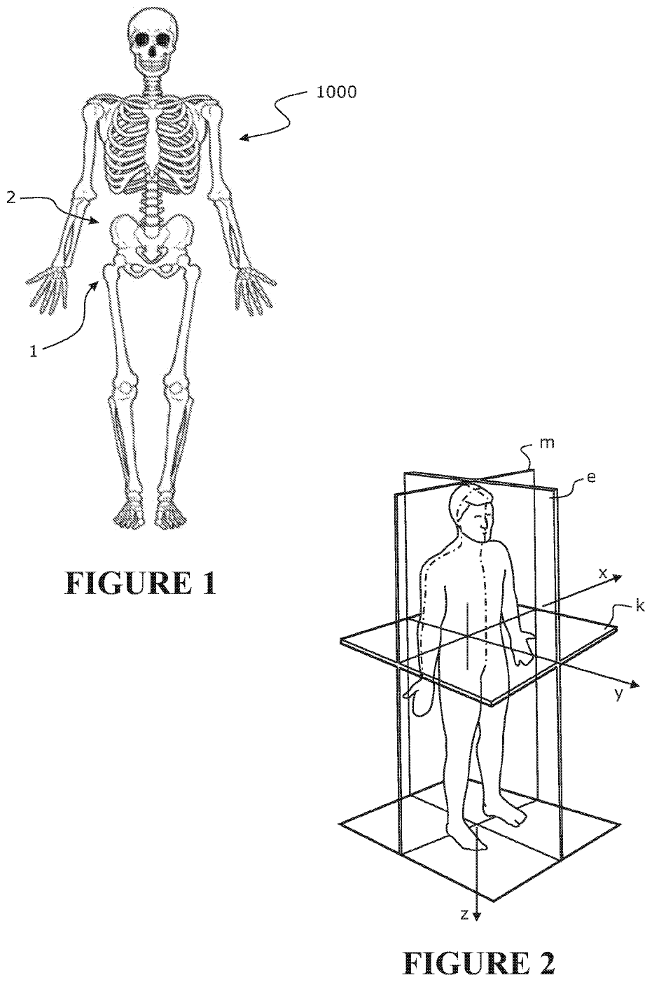

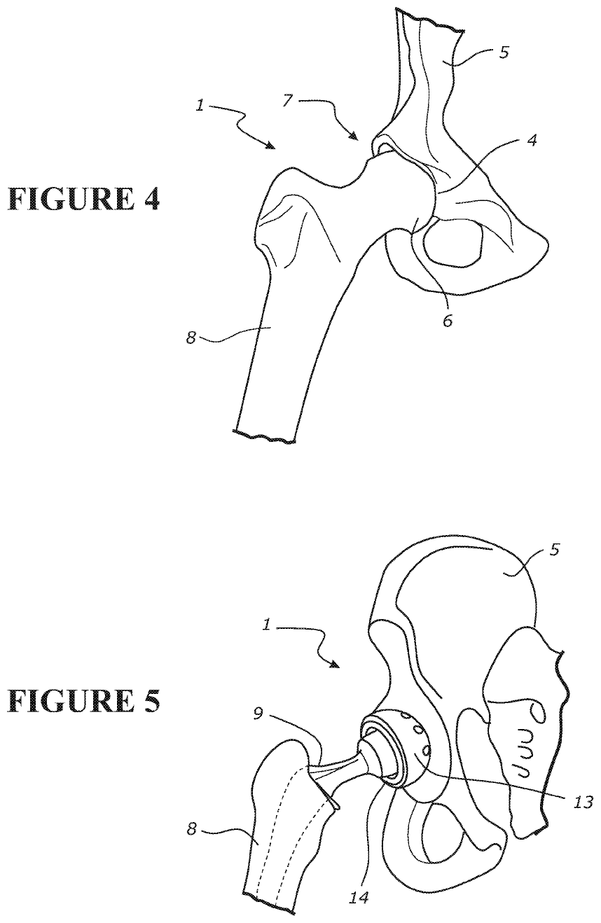

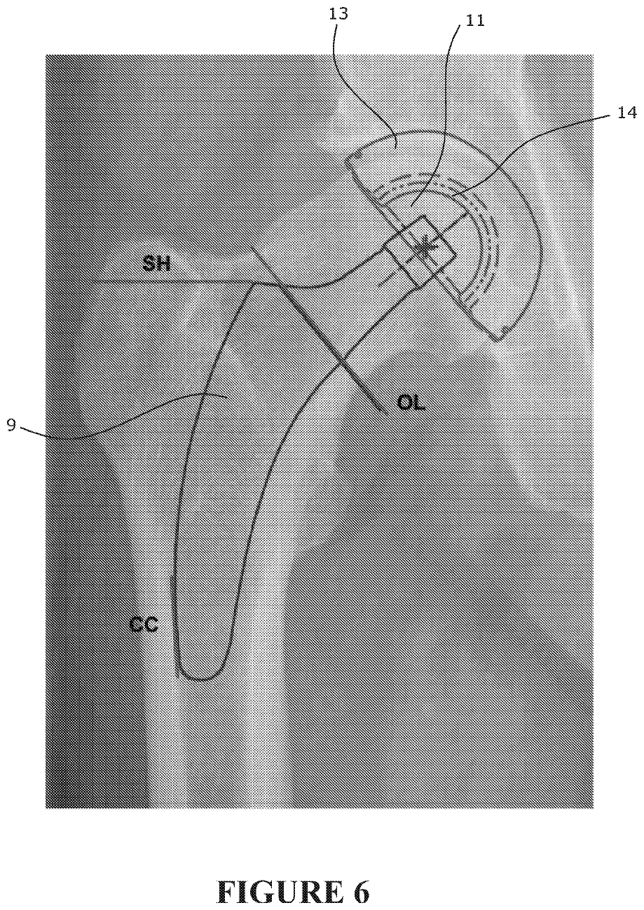

With reference to FIG. 1, which shows a human skeleton 1000, the hip joint 1 at the hip region 2 of a person can be considered a “ball in socket” joint. As seen in FIG. 4 the socket 4 is part of the pelvis 5 and the ball 6 is part of the femoral head 7 or the femur 8.

When a surgeon replaces a patient's natural hip with a hip prosthetic, they dislocate the hip joint 1 to release the ball 6 from the socket 4, remove the femoral head 7 of the femur and also remove the cartilage lining at the acetabulum (the socket) of the pelvis 5.

Cutting the bone of the femur 8 to remove the femoral head 7 is known as performing a “femoral osteotomy”. Removing the acetabular cartilage 12 is known as “reaming the acetabulum”, due to the acetabular reamer tool used to do this.

Once the femoral head 7 is removed, it is replaced by a metal stem 9 of the hip prosthetic 10. The metal stem 9 is pushed into the femur 8 to locate to the femur. At the end of the stem 9 that projects from the femur, a metal or ceramic ball 11 is presented. The acetabulum cartilage 12 is replaced typically with a hemispherical metal shell 13 of the hip prosthetic. This typically has a polyethylene cup 14 lining the inside. The metal or ceramic ball 11 can then be manoeuvred to locate to the cup to be able to articulate within the cup 14.

It is important that inter-operatively the surgeon puts the hip region back together so that the spatial relationship of the femur with the pelvis is the same post-op as it was pre-op. Or that it is put back together in a manner compensating for any arthritic degradation of the original hip region. If this does not occur there may be a resultant undesirable lengthening or shortening of the leg and/or a widening or narrowing of the hip. Such may affect the soft tissues surrounding the joint (muscles, tendons, ligaments, nerves, etc) so they may be too tight or too loose, either of which can cause a host of problems such as future dislocation, pain, nerve palsy and more.

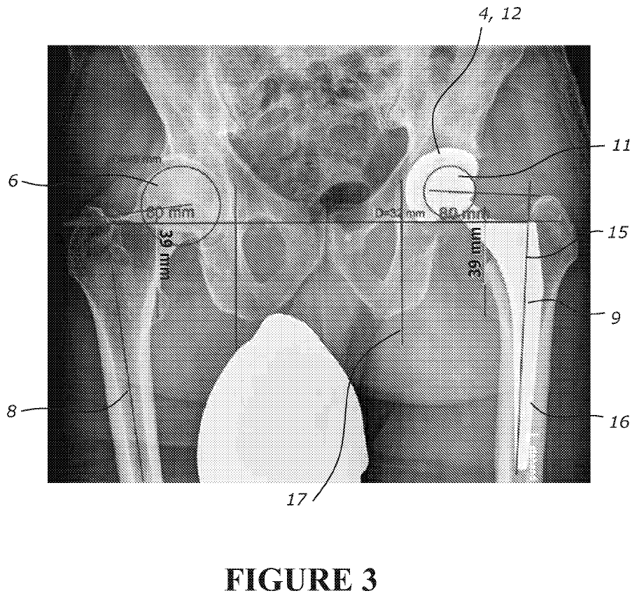

The spatial relationship is defined by geometry as described in practice by three measurements that are substantially at right angles to each other, namely:
1. Femoral Offset (FO)—this is the horizontal distance between the femur 8 and the pelvis 5. It is the offset parallel the X axis as seen in FIG. 2. Typically this is measured by determining the distance between two notional vertical lines, a first line that typically runs down the centre of the femoral shaft 16, and a second line that runs through a radiographic feature in the pelvis 5 known, due to its shape, as the “teardrop” as seen in FIG. 3. This offset distance is measured at the level of the bottom of the teardrop For example in FIG. 3 the femoral offset is shown as
2. Leg Length (LL)—this is the vertical distance between a fixed point on the femur and a fixed point on the pelvis. It is a linear measurement substantially parallel to the Y axis as seen in FIG. 2. For example in FIG. 3 the leg length can be compared using the distance from a landmark on the femur (the lesser trochanter) to a landmark on the pelvis (a horizontal line through both teardrops) which in this case is shown as 39 mm.
3. Anterior Offset (AO)—the anterior distance from the centre of the femoral head to the proximal femur axis in the sagittal plane (e) in the Y axis direction. The AO cannot be calculated from an Anterior-Posterior directed radiograph.

Once the femoral head has been removed, the desired LL, FO cannot be accurately set without the assistance of some means of measurement or referencing. AO has traditionally not been measured, possibly because surgeons are comfortable in achieving it through correct femoral anteversion (the angle between the femoral neck and the femoral shaft in the transverse plane ie how much forward of the shaft the femoral head is, toward the front of the body) and whilst versions of the tool of the present invention can assist in appropriate setting of AO a surgeon may not require guidance from the tool for setting of this variable. To help ensure the two (FO and LL) and preferably three (FO, LL and AO) measurements are set the same or desirably post-op compared to pre-op, only the before-to-after differences needs to be detectable by a surgeon inter-operatively. For example, the absolute leg length of the leg does not need to be measured, only a change in length needs be detected. The surgical guide tool 100 of the present invention provides a surgeon with a visual real time ability to detect the before-to-after differences.

The surgical guide tool 100 lends itself well to direct anterior approach total hip arthroplasty given the bone regions that are exposed or able to be exposed during surgery and once the soft tissue incisions have been made.

The tool does not rely on any tool dedicated invasive procedure. In one form the tool may rely on an initial cut being made of the neck of the head of the femur at the neck's anterior cortex only. This initial cut being the precursor of the full cut to remove the head of the femur as will hereinafter be described. The surgical guide tool 100 of the present invention provides an ability for a surgeon to visually detect, inter operatively, that the relative spatial disposition of the femur and the pelvis is restored or not, upon the coupling of the hip prosthetic's cup with its respective ball. It does so reliant on landmarks (natural and/or inter-operatively created) of and/on the upper femur and the pelvis to allow a visual comparison to be made between the before disposition (before the femoral head is removed) and after disposition (after the femoral head has been fully removed).

A preferred form and method of use of the tool 100 will now be described, followed by examples of variations that are within the scope of the invention.

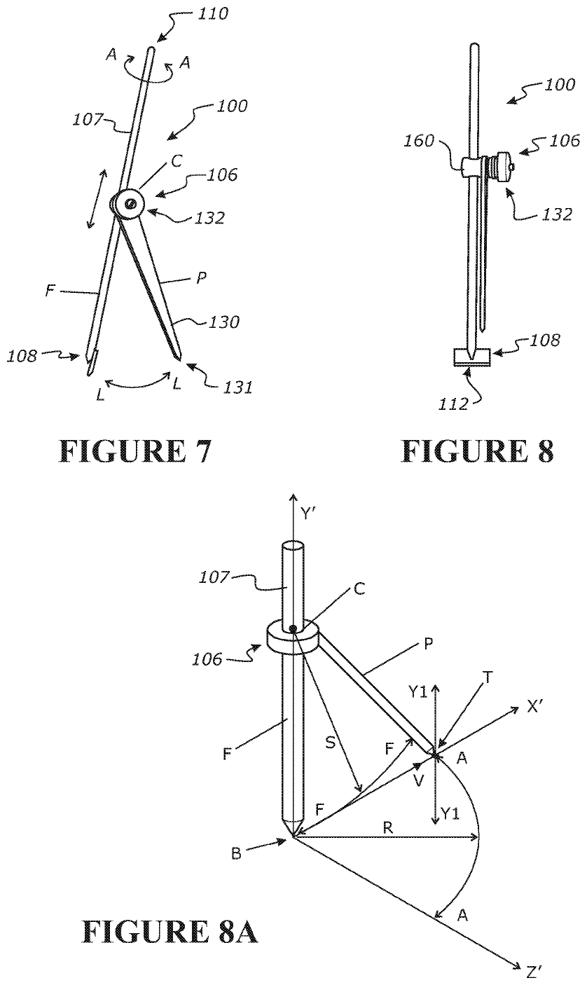

A preferred from of the surgical guide tool 100 is seen in FIGS. 7 and 8. The surgical guide tool 100 is able to visually aid in resetting of the pre-operative leg length (LL) and femoral offset (FO) and preferably the Anterior Offset (AO) of a patient inter-operatively during direct anterior approach arthroplasty.

The tool 100 comprises a femur contact member F able to make at least two point contact with the femur during hip surgery. This contact, as will hereinafter be described, will be at and/or below the femoral osteotomy cut 500 or where that cut will be made inter-operatively.

The purpose of the two point contact is to set a repeatable position and at least one notional angle of the femur contact member F on and relative to the femur 8. The two point contact is more than just establishing a mono-pod like relationship with the femur and instead sets at least a bi-pod relationship having a fixed position and a fixed angle. A tripod relationship may also be established as will be described with reference to variations of the tool to that shown in FIGS. 7 and 8.

The tool 100 as seen in FIGS. 7 and 8 also comprises a pelvis contact member P that is able to make at least one point contact with the pelvis 5.

The femur contact member (hereinafter “FCM”) and the pelvis contact member (hereinafter “PCM”) are coupled or able to be together. As seen in FIGS. 7 and 8 this coupling may occur at region 106 and in a manner that will hereinafter be described.

The FCM and PCM are preferably coupled together in manner able to move relative to each other or be otherwise set in different orientations relative each other. This is to allow the at least two point contact with the femur and the at least one point contact with the pelvis to be made. The at least two point contact is preferably first made with the femur followed by the one point contact with the pelvis. It is envisaged that the reverse may work where two point contact is first made with the pelvis and the one point contact with the femur. The use of the tool will in more detail herein after be described and it its preferred mode of use.

The nature of the coupling of the FCM and PCM allows for their relative movement to occur but is also able to prevent or lock the relative movement of the FCM to the PCM or to otherwise set their relative positions.

In use the two point contact of the femur by the FCM is at at least two spaced apart references respectively, on or of the femur. Such references are on or of the femur. As examples a femur reference may for example be a mark such as a line marked on the femur at or inferior of the femoral osteotomy cut location. A surgeon may for example mark a line at 500 on the surface of the femur at where the femoral osteotomy cut is to be made prior to making this cut. A femur reference may or may also be at least one mark X1 such as a dot or cross or V mark to create a visual point on the surface of the femur that is inferior of the femoral osteotomy cut location. A femur reference may or may also be at least one other mark, marked on the surface of the femur inferior of the femoral osteotomy cut location. The femur reference may or may also be the face 501 of the femur defined by the femoral osteotomy cut 500 or its anterior cortex precursor. The femur reference may or may also be a clearly determinable natural landmark established as a point mark on the surface of the femur inferior of the femoral osteotomy cut location. It should be noted that natural landmarks are unlikely to be as clearly defined as an introduced landmark.

Preferably in the most preferred form the references on or of the femur are (a) at least one mark X1 such as a dot or line marked on the femur inferior of the femoral osteotomy cut location, and (b) the face 501 of the femur defined by the femoral osteotomy cut (or its anterior cortex precursor) or a line marked on the femur where the femoral osteotomy cut will be made. The mark X1 may instead be a line as seen in FIG. 17.

As seen in FIGS. 7 and 8, preferably the FCM (F) comprises of a leg 107 (herein after “femur leg”). It is preferably of metal such as stainless steel. It is preferably elongate and straight and preferably substantially of constant cross section along its length. It is preferably of a circular circumferential cross section.

The femur leg 107 has a first distal end 108 at where the at least two point contact with the femur can occur. Preferably the at least two point contact for the FCM is defined by two spaced apart contact members or regions of the FCM. A plate 109 and a point B (preferably defined by a pin) is preferably provided at the first distal end 108. The plate 108 is preferably secured to the femur leg 107 in a fixed manner (eg it may be welded or otherwise bonded or secured to the femur leg or integrally formed therewith) and point B is preferably defined by a pin that is at the end of the femur leg 107. As can be seen in FIG. 23 point B is offset from the plate 109. The pin may be part of the femur leg (eg formed at its distal end) of engaged to the femur leg.

The plate has a femoral contact surface 111 that is preferably planar. The surface 111 is able to locate against and parallel the cut surface 105 of the femur 8 during use. The method of use will hereinafter be described in more detail. The contact between the surface 111 of the plate and the cut surface 105 of the femur allows for the tool to register with the femur. The plate 109 may also include an edge 112. The edge allows for the tool to be placed on a line that may be marked on the femur. This allows the edge and hence the tool to register with the femur. This line may be the guide line for the femoral osteotomy cut also. Or a separate line. Hence in use the plate may be used to either register with the cut surface 105 of the femur or with a line marked on the femur. This registration sets a rotational position of the femur leg with and relative to the femur.

Axes X′ Y′ and Z′ as seen in FIG. 8A (which shows a schematic view of the tool but without the plate being shown) are an offset variation of axes X, Y and Z respectively and seen in FIG. 2, offset setting the axis X′ to extend in a direction that is Normal to the plane of the cut 501 of the femur.

The plate and its registration with the surface 501 or with a line marked on the femur allows for the leg 107 to rotationally (about notional axis Y′) register with the femur. This registration positions the FCM rotationally relative to the femur. For ensuring that the FCM also registers (not just rotationally but also to a point) of the femur, the point B can be used to register with a mark such as mark X1 that a surgeon may place and mark on the surface of the femur adjacent to the cut 501.

The point B, offset from the surface 111 on the inferior side of the cut 501, can be used to align the FCM with the mark X1 on the femur. Or for mark X1 to be made corresponding to where point B is placed. Both the rotation about the axis Y′ and the position of the FCM relative to the femur is then able to be set.

The plate is preferably of a thickness no thicker than the saw used to make the cut. This allows the plate to slip into the initial cut that is made into the femoral neck. The plate need only slip into the femoral neck slightly in order to for it to help set the angle about axis Y′.

The mark X1 may instead be a line as seen in FIG. 17 and can be landed on by the point B with the plate registered against the surface 501 to position the FCM rotationally and positionally relative the femur. The provision of the line X1 as seen in FIG. 17 is the preferred way for setting the position of the tool relative the femur. A visual determination of rotation of the femur leg 107 about axis Z′ and axis X′ may be able to be visually determined and set sufficiently accurately by the surgeon during use of the tool. However, alternative forms of the tool of the present invention are herein described to help set and repeat the rotation of the femur leg 107 about axis X′ and Z′ also.

Whilst in FIG. 8A the femur leg 107 is shown extending parallel to and on the axis Y′ it will be appreciated that it may be in use at an angle, as the axis Y′ is merely a notional angle for illustrative purposes only.

In the preferred form the femur leg 107 is elongate and straight. It is preferably circular in peripheral cross-section or at least circular in cross-section at where the coupling at region 106 is made. This will hereinafter be described.

The PCM (P) preferably comprises of a leg 130 (hereinafter “pelvic leg”). The pelvic leg 130 is preferably elongate and straight as seen in FIGS. 7-9 and has a first distal end 131 and a second distal end 132 at the coupling region 106. At the first distal end 131 the pelvic leg 130 presents a point T. Point T may be defined by a sharp end of the distal end 131 of the pelvis member 130. As can be seen in FIG. 11, this point T is able to register with the mark X2 on the pelvis. In a preferred form the mark X2 is at the anterior inferior iliac spine (AIIS) which is a bony landmark on the pelvis easily visible to the surgeon during surgery. A surgeon is able to place the mark X2 on the pelvis at this region during surgery and at a time of the surgery as will hereinafter be described.

At its second distal end 132 the pelvis leg 130 is coupled to the FCM, preferably directly or indirectly to femur leg 107. The coupling allows for the position of the point T to be adjusted relative to point B of the tool, such position preferably able to be adjusted is both in distance and in rotation.

As seen with reference to FIG. 8A the PCM (P) is able to rotate about axis Y to be able to move in the direction of the arc AA at a radius R from point B. Radius R is preferable adjustable also as will be described though in the example shown in FIG. 26 that is not so.

In a preferred form this rotation in the direction of arc AA is facilitated by the fact that the femur leg 107 at least at the coupling region may be circular in cross-section and the coupling may comprise of a sleeve barrel with a circular hole 161 to snugly locate about the femur leg 107.

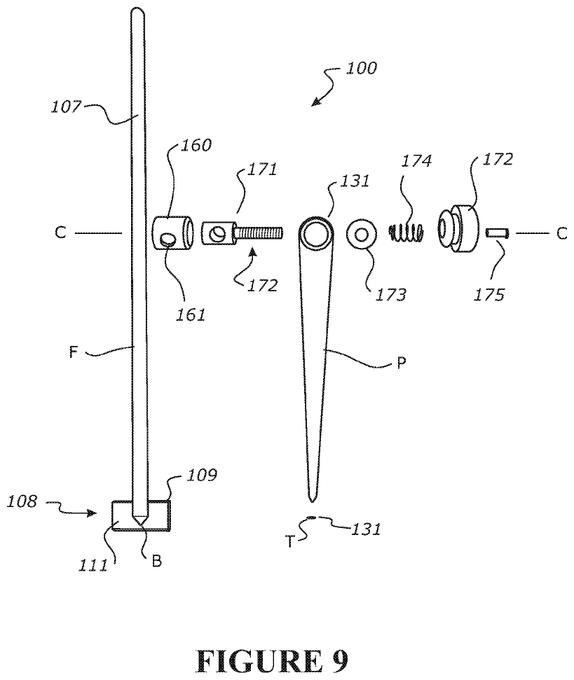

In the exploded view in FIG. 9, a bolt 171 with a threaded portion 172 can pass into the barrel 160 and also be received to the tightening nut 172. The tightening nut 172 can threadingly engage onto the thread 170 and draw the bolt 171 and move along the bolt in a manner so as to loosen and tighten the coupling 106. This allows for the coupling to (a) locked for the purposes of retaining the vector V and (b) loosen to allow for the vector V to point T from the distal end 108 of the FCM to be adjusted. A washer 173 and spring 174 may also be utilised in the assembling of the coupling. In addition, a lock screw 175 may be used to help prevent the coupling from coming apart, though this may be undone to allow the coupling parts to come apart to allow the parts to be fully sterilised.

The coupling preferably also allows for the PCM to rotate relative to the FCM such as about point C so that point T can travel in a radial direction relative to point B such as parallel arc LL about point C at a radius S. The coupling at the coupling region 106 preferably provides an ability to rotate about axis CC. Whilst in a preferred form the distance between T and B is able to be varied, this distance may instead be permanently set. Hence a rotation of the PCM relative the FCM about axis CC may not be essential.

In addition, preferably the coupling is able to axially displace along the femur leg 107 between its distal ends 110 and 108. Preferably the PCM is able to translate relative the FCM.

Hence as seen in FIG. 8A the tool is able to adjust a vector V from point B to point T in a 3 dimensional reference frame. The movement allowed by the coupling in this manner, and the rotation about the axis Y′ and the radial displacement along arc LL about axis CC, allows for a vector V to be set for point T relative to distal end 108 of the FCM. The range of the vector V is sufficiently large for the tool to be used during multiple direct anterior approach surgeries having different distances between point B and point T.

In some forms, the tool may have a set distance between points A and B based on the fact that it is possible to mark a point X1 and a point X2 on the femur and pelvis respectively that is of a fixed distance. This may mean that radius R may be fixed yet rotation of the pelvis leg relative the femur leg about axis Y′ may still be facilitated. A tool with this configuration is shown in FIG. 26 as an example. Likewise relative rotation along arc AA may not be possible by a tool that is a variation of the preferred form of the invention. The angle between the plate and point T may hence be fixed as an example and the other degree or degrees of freedom of movement of point T relative the plate may be provided for.

Points B, B1, B2 etc and T may be defined by pins.

Reference will now be made to the manner of use of the surgical guide tool 100. As has been mentioned the surgical guide tool 100 is for use during direct anterior approach total hip arthroplasty.

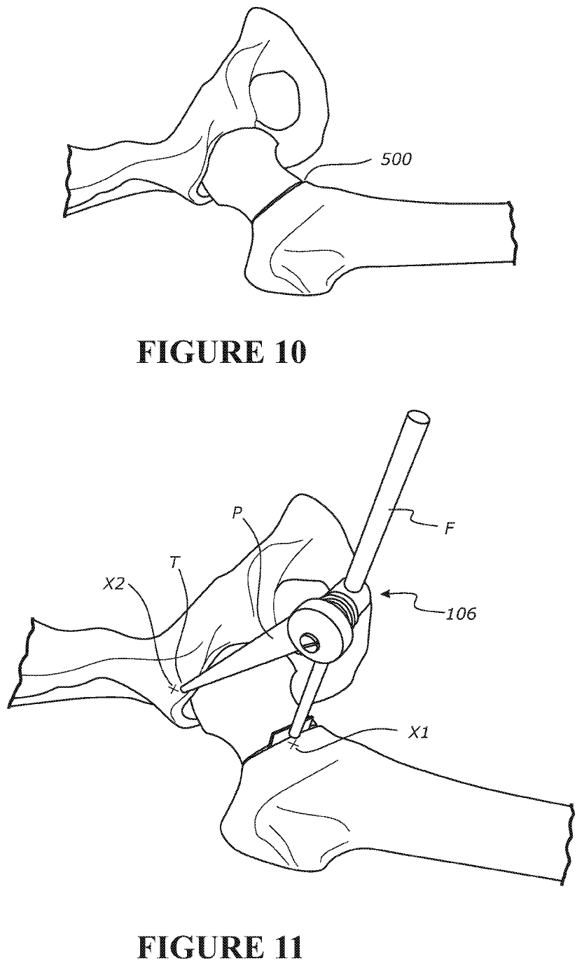

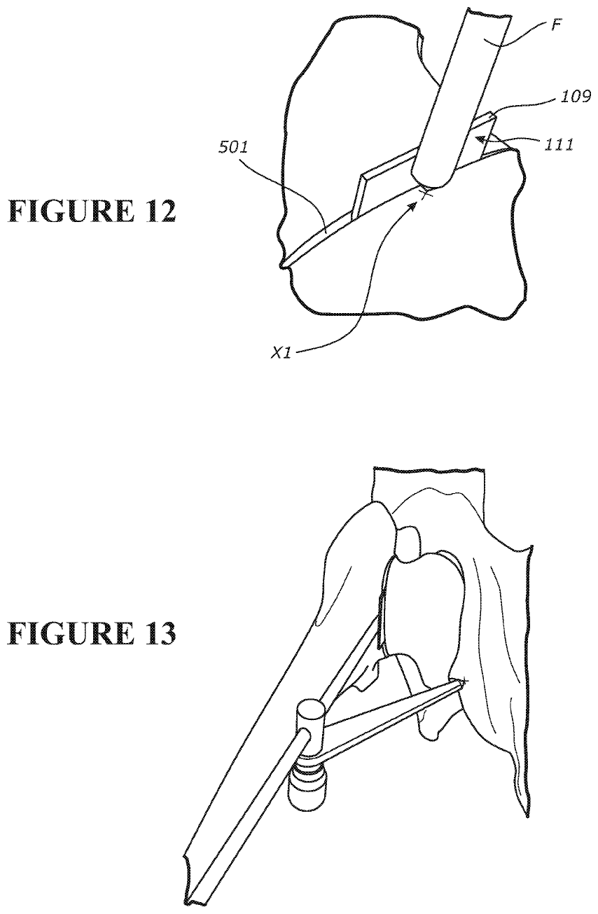

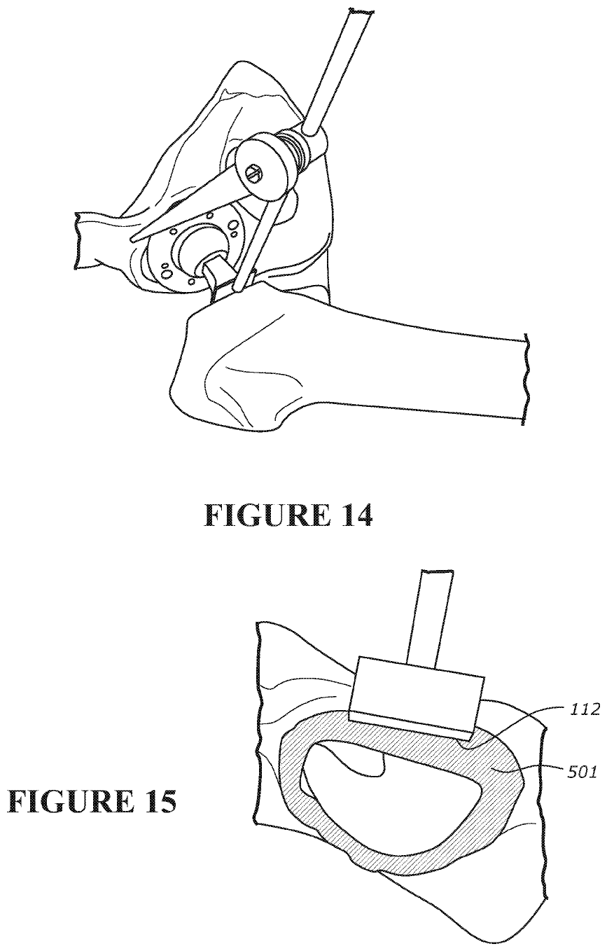

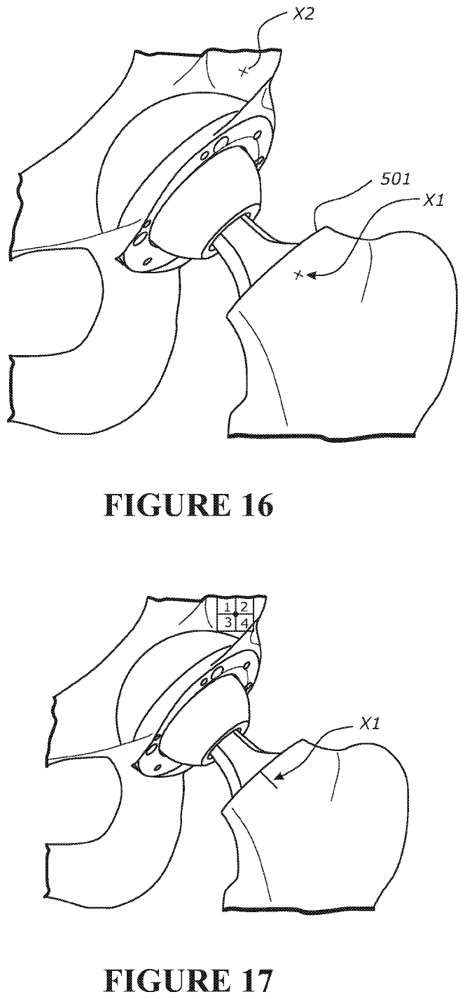

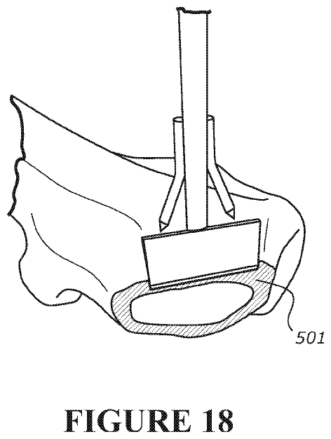

In a preferred form once the soft tissue has been cut to gain access to the hip joint, a surgeon may either mark the position of the femoral osteotomy with a line 500 on the femoral neck (e.g. using a surgical marker pen or diathermy) or begin the osteotomy but only cut the neck's anterior cortex only. The line or cut are provided to allow for the plate 109 to register with the cut surface or at line. If it is a line, the edge 112 of the plate is able to locate on and alight with the line. If it is a partial cut of the femoral neck at the anterior cortex, as seen in FIGS. 10-12, the face 111 of the plate 109 is able to align with and register against the cut surface 105 of the femur. This line of cut allows an angle of the plate and hence the femur leg 107 of the tool 100 to be set, that can then be repeated.

A surgeon can also make a first mark such as mark X1 which may be in the form of a “.” or an “X” or a “V” or a line as seen in FIG. 17, on the femur inferior of the cut, such as with a pen or diathermy. This may occur before or after the line 500 or cut 500 has been created. The mark X1 may be on the relatively flatter region/surface at the anterior of the femoral neck. This mark may be made such as a few millimetres inferior of the cut or line 500 at where the cut will be made. The mark X1 provides a mark acting as a positional reference for distal end of the FMC (such as for point B) relative the femur and for the FCM to be able to repeatably register with. The mark X1 may be made before the point B is placed thereat or whilst point B is placed there at. Alternatively it may be made immediately after point B has been placed thereat (having preferably left a pilot mark to guide the surgeon to place mark X1 at the pilot mark).

A second mark such as mark X2 may be made by the surgeon on the anterior inferior iliac spine, such also being easily visible to the surgeon during the procedure. The appropriate location of mark X2 may be determined by the surgeon reliant on good judgement taking account that point T is to make contact with mark X2 in due course.

With the references (such as the mark(s) and/or cut surface) on or of the femur and with the reference mark on the pelvis, the tool can be positioned at the surgical site. This can occur by placing the plate aligned on the line 500 or against the cut surface 501 and point B to register at the mark X1. This then sets an angle of the FCM and location thereof at the femur. The PCM is then able to be manoeuvred by the surgeon by virtue of the coupling being sufficiently flexible or lose to allow for point T of the PCM to be located at the mark X2. The coupling may then be locked so that the distance and rotational angle setting of point T relative to point X1 and the angle of the plate, still set at the femur side of the hip region, can be set. The angles and distance of point T so set 3 dimensionally, may herein referred to as vector V.

It is envisaged that in some methods of use, point T may be used to define the location of mark X2 prior to femoral head removal and whilst the FCM is registered in both position and an angle of rotation relative to the femur. This would be instead of marking mark X2 first and then setting point T to it.

Once the FCM and PCM are locked to each other to prevent inadvertent change in the set vector V, the tool can then be removed from the surgical site. The femoral head may then be completely removed.

After reducing the hip with trial implants the FCM is able to be placed back at its original position as seen in FIG. 14. Mark X1 remains and so does sufficient of the cut surface 501 of the femur. Point B can be placed on mark X1 and surface 111 of the plate 109 is able to register against the surface 501 to set both the original rotational angle and location of the FCM relative to the femur. A surgeon is able to visually ensure that the femur leg 107 projects at a repeated angle from the femur, such being preferably substantially at 90 degrees to the relatively flatter surface of the femoral neck. In other forms of the tool described below one of both of the other orthogonal angles may also be settable by the tool.

With the rotational position about axis Y′ of the femur leg 107 being set by virtue of the plate registering with the surface 501 (of a line on the femur) of the femur and point B being positioned at X1, the distance R between points B and point T having been set prior to the full removal of the femoral head, point T of the PCM can now be used to detect whether point X2 is at the same position as before the operation.

Should point T land on mark X2 (with the plate and point B registered appropriately at the femur) then a surgeon will know that the femoral offset and leg length has been repeated. If point T does not land on point X2 then one or both of the FO and LL has not been repeated.

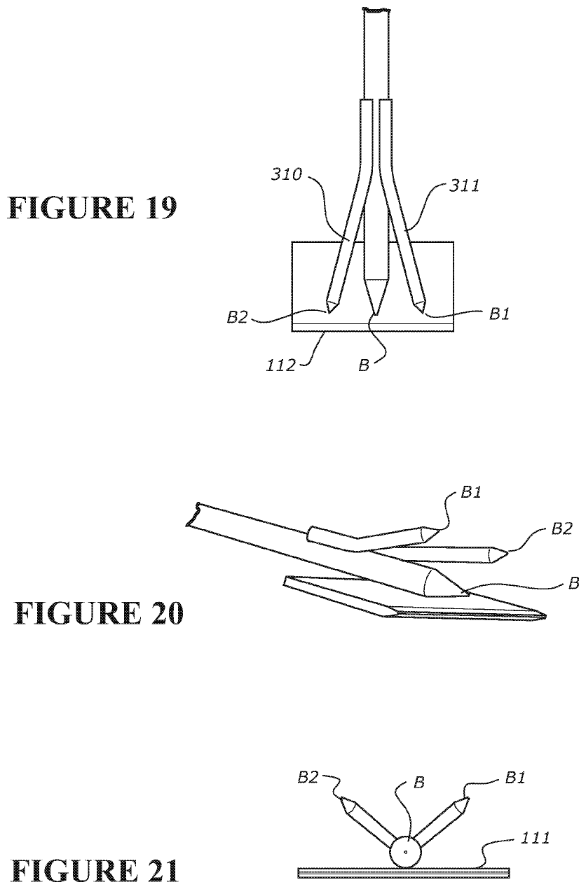

A visual positioning of the femur leg 107 to ensure its repeat in angular position about axis X′ and axis Z′ may be visually achieved by a surgeon sufficiently accurately. In any event any slight deviation from original angles about X′ and Y′ is not going to make a substantial difference in a repeating of the location of point T post femoral head removal for LL and FO setting. However, in order to ensure that more than one angle, not just about axis Y′, but also about axis X′ and/or Z′ is repeatable by the FCM, additional features in alternative forms of the present invention may be included and these will now be described with reference to FIGS. 19-22.

As can be seen in FIGS. 19-21 showing a variation of the tool, the FCM comprises additional members presenting points B1 and B2. The FCM presents points B1 and B2 from tripod legs 310 and 311 that are secured the femur leg 107. They may be permanently secured and rigidly secured to (or integrally formed with) the femur leg 107 and present points B2 and B1 in a manner to allow for the FCM to be supported in a tripod manner on the femur, inferior of the cut 500. The provision of a tripod-like arrangement at the distal end 108 of the FCM allows for the FCM to repeat its angular position in each of the orthogonal axis relative the femur. The mark X1 may be provided for contact by point B or by any of points B1, B2 or B.

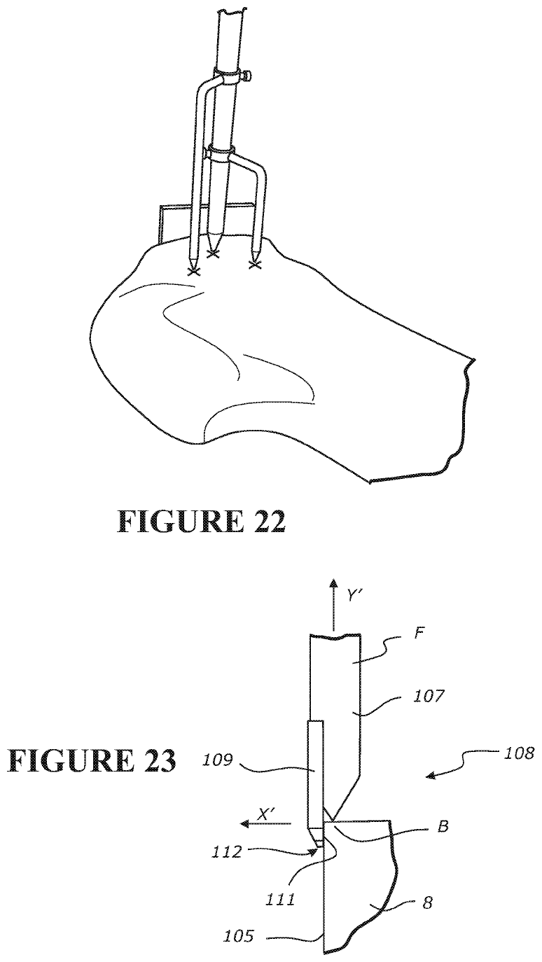

In a variation as seen in FIG. 22, one or both of the tripod legs may be dependent from the femur leg 107 in a manner to allow for one of both the axial position and the angular position of their respective points to be adjusted relative to point B. A sliding and/or rotatable coupling that can be loosened and locked relative to the femur leg 107 may be provided for such purposes.

The provision of a tripod-like arrangement at the distal end 108 of the FCM will help enhance the accuracy of the tool as a guide for surgeons in restoring the FO and LL and AO to their pre-operative condition.

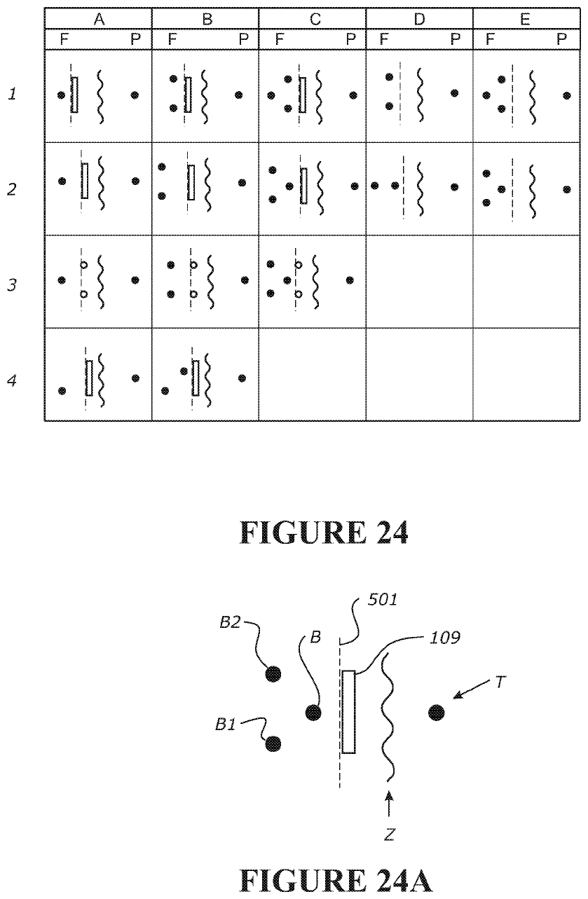

With reference to FIG. 24A there is shown a schematic of one arrangement of the tool of where the line Z is a notional break line between the femur side and the pelvis side, also showing point B, B1, B2, point T and the plate 109 and cut face 501 represented by the dotted line. Other variations of points B and/or B1 and/or B2 and/or the plate 109 and point T are schematically illustrated in the table of FIG. 24 showing examples of their dispositions on the femoral side (F) and the pelvis side (P).

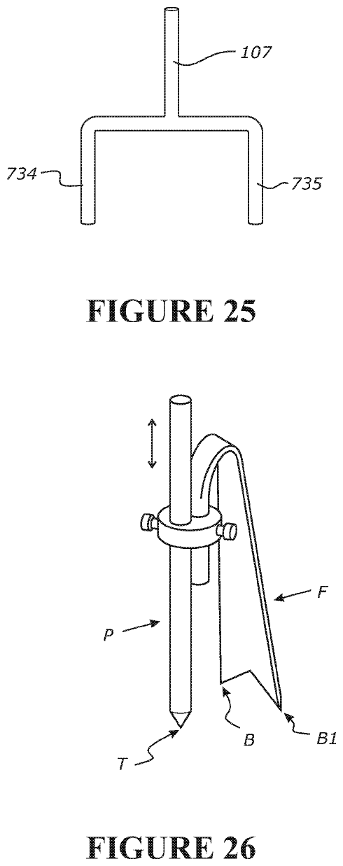

The provision of two points, B and B1 by the FCM helps improve the accuracy of the angular position of the tool, compared to the single point B version as seen in FIG. 11 for example. Whilst in the preferred form a plate 108 is provided to make at least two point contact with the femur for primary angular positioning of the FCM relative to the femur, instead the plate may be substituted by two bars 734 and 735 to provide for spaced apart two-point contact on the cut surface 501 of the femur. Such two-point bar contact is shown in row 3 of the table of FIG. 24 and is as for example seen in FIG. 25. In FIG. 25 there is shown the two bars 734 and 735 disposed at the end of the femur leg 107 of the FCM.

Other variations for the coupling as shown in FIG. 9 are also envisaged. The coupling could be provided as a ball and socket connection or other flexible yet lockable connection. The range of movement provided by the preferred form of coupling could instead be provided by two or more separated couplings.

A further alternative to the preferred form of the invention to help illustrate the broad concept of the invention is shown in FIG. 26. This shows a tool that may correspond to the configuration schematically shown in box D1 of FIG. 24. In this example a two point contact is able to be made with the femur by points B and B1 to set position and an angle of rotation of the FCM (F) relative to the femur. The geometry of the tool may be such that point T and B and B1 are able to contact the pelvis and femur respectively at appropriate locations to not require a rotational adjustment to be made between the PCM and the FCM. Optionally the PCM may be mounted to the FCM to translate in the directions as indicated by the arrow.

Other variations to allow adjustment of the vector V may include a telescopic femur leg and/or telescopic pelvis leg and/or an intermediate member between the femur leg and pelvis leg.

The tool is preferably reusable. The tool is not intended for single use. The tool is able to be sterilised conveniently. It is preferably made of metal components and is able to be easily disassembled and reassembled. The tool is not intended to be invasive or used invasively, noting that the cut surface 501 that it may rely on to reference to is being made for femoral head removal and not purely for tool use. The tool is not an active device.

The tool is preferably a single product and able to so be used during hip replacement surgery to help detect a difference in FO, LL and preferably AO during direct anterior approach total hip arthroplasty. It does not require other tools or components or instruments for its effective use for such purposes, during surgery.

The tool is able to set the vector V, as hereinbefore described, mechanically. No electronics components are required for its effective use. The tool is used inter-operatively and preferably for direct anterior approach hip replacement surgery.

Where in the foregoing description reference has been made to elements or integers having known equivalents, then such equivalents are included as if they were individually set forth.

Although the invention has been described by way of example and with reference to particular embodiments, it is to be understood that modifications and/or improvements may be made without departing from the scope or spirit of the invention.

In addition, where features or aspects of the invention are described in terms of Markush groups, those skilled in the art will recognise that the invention is also thereby described in terms of any individual member or subgroup of members of the Markush group.

1. A surgical guide tool to visually aid in setting of the desired leg length and femoral offset of a patient inter-operatively during direct anterior approach arthroplasty, the tool comprising:
a. a femur contact member able to make at least two point contact with the femur at and/or inferior the femoral osteotomy cut, to set a position and at least one notional angle of the femur contact member on and relative the femur, and
b. a pelvis contact member able to make at least one point contact with the pelvis,
where the femur contact member and the pelvis contact member are coupled together;
(a) in manner able to move relative to each other to allow (i) said at least two point contact with the femur and (ii) said at least one point contact with the pelvis to be made, and
(b) in a manner to lock relative movement of the pelvis contact member and femur contact member.

2. A tool as claimed in claim 1 wherein the at least one notional angle is an angle about an axis that extends normal to the coronal plane of the patient.

3. A tool as claimed in claim 1 wherein the at least one notional angle is an angle about an axis that extends normal to the transverse plane of the patient.

4. A tool as claimed in claim 1 wherein the at least one notional angle is an angle about an axis that extends normal to the sagittal plane of the patient.

5. A tool as claimed in claim 1 wherein said at least two point contact of the femur by the femur contact member is at at least two spaced apart references respectively, on or of the femur.

6. A tool as claimed in claim 5 wherein the references on or of the femur are selected from any two of:
a. a line marked on the femur at or inferior of the femoral osteotomy cut location,
b. at least one mark marked on the femur inferior of the femoral osteotomy cut location,
c. at least one other mark marked on the femur inferior of the femoral osteotomy cut location,
d. the face of the femur defined by the femoral osteotomy cut or its anterior cortex precursor, and
e. a natural landmark of the surface of the femur inferior of the femoral osteotomy cut location.

7. A tool as claimed in claim 5 wherein the references on or of the femur are (a) at least one mark marked on the femur inferior of the femoral osteotomy cut location, and (b) the face of the femur defined by the femoral osteotomy cut or its anterior cortex precursor.

8. A tool as claimed in claim 1 wherein said at least one point contact of the pelvis contact member is at at least one reference on or of the pelvis.

9. A tool as claimed in in claim 8 wherein the at least one reference on or of the pelvis is selected from one of:
a. a line marked on the pelvis,
b. a point marked on the pelvis, and
c. a natural landmark of the pelvis.

10. A tool as claimed in claim 1 wherein the at least two point contact for the femur contact member is provided by two spaced apart contact members of the femur contact member.

11. A tool as claimed in claim 1 wherein the femoral contact member comprises of a leg (hereinafter “femur leg”) having a first distal end at where the at least two point contact for the femur contact member directly or indirectly occurs.

12. A tool as claimed in claim 11 wherein the two spaced apart contact members are presented at the end of the femur leg.

13. A tool as claimed in claim 11 wherein a first of said two spaced apart contacts members is a pin.

14. A tool as claimed in claim 13 wherein the pin is defined by and at the end of the femur leg.

15. A tool as claimed in claim 11 wherein a second of said two spaced apart contact members is a plate having a surface or surfaces able to be held against the cut surface of the femur or the line mark of the femur to align therewith.

16. A tool as claimed in claim 15 wherein the plate is attached to the femur leg.

17. A tool as claimed in claim 15 wherein the plate is attached to the femur leg in a fixed manner.

18. A tool as claimed in claim 15 wherein the plate, when located against the cut surface or the line marking on the femur, sets a said notional angle of the femur contact member relative the femur.

19-33. (canceled)

34. A surgical guide tool for interoperative use during direct anterior hip replacement surgery to aid in detecting a change in LL and FO at the surgical site during final prosthetic configuration to the patient, the tool comprising:
a. a femur contact member able to positionally and rotationally register on and with the femur by provision of at least two femur contact points of the femur contact member,
b. a pelvis contact member able to positionally register on and with the pelvis,
wherein the pelvis contact member and the femur contact member are (a) coupled together by a coupling in (a) a movable manner relative each other so that the pelvis contact member can positionally register with a reference on the pelvis when the femur contact member is positionally and rotationally registered with respective references of the femur prior to total femoral head removal and (b) a manner able to prevent movement between the pelvis contact member and the femur contact member so that after total femoral head removal the femur contact member can be returned to the surgical site and register at the references of the femur to visually detect change in LL and FO.

35. A surgical guide tool for interoperative use during direct anterior hip replacement surgery at the surgical site to aid in setting LL and FO during final prosthetic configuration to the patient, the tool comprising:
a. a femur contact member able to positionally and rotationally register on and with the femur by provision of at least two femur contact points of the femur contact member,
b. a pelvis contact member able to positionally register on and with the pelvis,
wherein the pelvis contact member and the femur contact member are (a) coupled together by a coupling in (a) a movable manner relative each other so that the pelvis contact member can positionally register with a reference on the pelvis when the femur contact member is positionally and rotationally registered with respective references of the femur prior to total femoral head removal and (b) a manner able to prevent movement between the pelvis contact member and the femur contact member so that after total femoral head removal the femur contact member can be returned to the surgical site and register at the references of the femur to visually detect change in LL and FO.

36-40. (canceled)
